(19) United States
(12) Patent Application Publication
(10) Pub. No.: US 2017/0343515 A1
(43) Pub. Date: Nov. 30, 2017

(54) APPARATUS AND METHOD FOR OBTAINING OBJECT INFORMATION AND NON-TRANSITORY COMPUTER-READABLE STORAGE MEDIUM

(71) Applicant: CANON KABUSHIKI KAISHA, Tokyo (JP)

(72) Inventor: Hiroshi Abe, Yokohama-shi (JP)

(21) Appl. No.: 15/679,781

(22) Filed: Aug. 17, 2017

Related U.S. Application Data
(63) Continuation of application No. 13/758,142, filed on Feb. 4, 2013.

(30) Foreign Application Priority Data
Feb. 7, 2012 (JP) 2012-024141

(51) Int. Cl. G01N 29/24; A61B 5/00; A61B 8/08

(52) U.S. Cl. CPC G01N 29/2418; A61B 5/0095; G01N 2291/02466; A61B 8/485; G01N 2291/02827; G01N 2291/018

(57) ABSTRACT

An object information obtaining apparatus includes a signal processing unit configured to obtain weighted optical characteristic information about an object on the basis of feature information about the object obtained by elastography measurement or B-mode image measurement using an elastic wave signal acquired by transmission and reception of elastic waves to and from the object.

### CROSS-REFERENCE TO RELATED APPLICATIONS

This application is a Continuation of co-pending U.S. patent application Ser. No. 13/758,142, filed Feb. 4, 2013, which claims foreign priority benefit of Japanese Patent Application No. 2012-024141 filed Feb. 7, 2012, all of which are hereby incorporated by reference herein in their entirety.

### BACKGROUND OF THE INVENTION

### Field of the Invention

The present invention relates to an object information obtaining apparatus for obtaining optical characteristic information using photoacoustic waves generated by irradiation of an object with light.

### Description of the Related Art

Development of optical imaging systems for irradiating a living subject with light emitted from a light source, such as a laser, and imaging information about the inside of the living subject obtained on the basis of incident light are being advanced in the medical field. One of such optical imaging techniques is photoacoustic imaging (PAI). In photoacoustic imaging, a living subject is irradiated with pulsed light emitted from a light source, photoacoustic waves (typically, ultrasonic waves) generated from biological tissue which has absorbed the energy of the pulsed light propagated and diffused inside the living subject are received, and optical characteristic information about the inside of the living subject is imaged on the basis of detection signals obtained from the received waves.

Specifically, photoacoustic imaging uses the difference between the absorptance of optical energy of tissue in a target site, for example, a tumor, and that of another tissue. A probe (also called a transducer or acoustic wave detector) receives photoacoustic waves (typically, ultrasonic waves) generated from the tissue in the target site upon instantaneous expansion of the tissue which has been irradiated with light and absorbed the energy of the light. Detection signals obtained from the received waves are analyzed, thus obtaining optical characteristic information. Herein, the optical characteristic information includes an initial sound pressure, an optical absorption energy density, or an optical absorption coefficient. The optical characteristic information further includes a distribution of such parameters.

In addition, the optical characteristic information includes the concentration of a substance (for example, the concentration of hemoglobin in blood or the saturation of oxygen in the blood) inside an object obtained by measurement using light of different wavelengths.

There are various image reconstruction methods for forming an image on the basis of detection signals obtained through a probe. To analyze a distribution of initial sound pressures of photoacoustic waves on the basis of detection signals obtained through the probe is typically called analysis of an inverse problem. In photoacoustic imaging, solving a photoacoustic wave equation under ideal circumstances proves that the inverse problem has a unique solution. As an example, an analytical solution of universal back projection (UBP) that represents the result of analysis in the time domain is as follows.

$${p}_{0} ( {r}^{->} ) = \frac{2}{{Ω}_{0}} {\int }^{ } {[ p ( {{r}_{0}}^{->} , t ) - t \frac{\partial }{\partial t} p ( {{r}_{0}}^{->} , t ) ]}_{t = \vert {r}^{->} - {{r}_{0}}^{->} \vert / {v}_{s}} d {Ω}_{0} \qquad ( 1 )$$

p₀(\vec{r}): the initial sound pressure distribution
p(\vec{r₀},t): the detection signal
dΩ₀: the solid angle for the probe with respect to an observation point

As described above, according to UBP, a detection signal p(r₀, t) obtained through the probe and detection signals differentiated with respect to time are subjected to solid angle correction (correction by a measurement system) and the results are added, thus obtaining the initial sound pressure distribution p₀(r) (refer to PHYSICAL REVIEW E 71, 016706 (2005)).

The method disclosed in PHYSICAL REVIEW E 71, 016706 (2005) has the following disadvantages.

Since the photoacoustic wave equation is solved under ideal circumstances, the conditions include a condition which could not be actually realized. For example, although a solution can be obtained under a situation where acoustic wave detecting elements are arranged in one plane in the above-described UBP, an ideal solution is obtained on condition that the arrangement is infinitely unlimited. Actually, however, the number of acoustic wave detecting elements arranged is limited. Information is obtained in only regions equal in number to the arranged acoustic wave detecting elements. Consequently, an artifact may occur in a reconstructed image. If an artifact occurs in the boundary between a region of interest and another region in a photoacoustic image, the contrast between the region of interest and the other region in the photoacoustic image will be reduced.

Furthermore, if a noise image caused by system noise occurs in the boundary between a region of interest and another region in a photoacoustic image, the contrast ratio of the region of interest to the other region will be reduced.

### SUMMARY OF THE INVENTION

The present invention provides an object information obtaining apparatus for obtaining a photoacoustic image with high contrast between a region of interest and another region using photoacoustic imaging.

According to an aspect of the present invention, an object information obtaining apparatus includes a signal processing unit configured to obtain weighted optical characteristic information about an object on the basis of feature information about the object obtained by elastography measurement or B-mode image measurement using an elastic wave signal acquired by transmission and reception of elastic waves to and from the object.

Further features of the present invention will become apparent from the following description of exemplary embodiments with reference to the attached drawings.

### BRIEF DESCRIPTION OF THE DRAWINGS

FIG. 1 is a schematic diagram of an object information obtaining apparatus according to an embodiment of the present invention.

FIG. 2 is a flowchart of a method for obtaining object information according to the embodiment.

FIG. 3A is a front view of an object and a probe in the embodiment.

FIG. 3B is a diagram illustrating feature information in the embodiment.

FIG. 3C is a diagram illustrating a photoacoustic wave signal to be corrected in the embodiment.

FIG. 3D is a diagram illustrating a weighted photoacoustic wave signal in the embodiment.

FIG. 4A is a diagram illustrating an initial sound pressure distribution obtained by the object information obtaining apparatus according to the embodiment.

FIG. 4B is a diagram illustrating a distortion distribution obtained by the object information obtaining apparatus according to the embodiment.

FIG. 4C is a diagram illustrating a weighted initial sound pressure distribution obtained by the object information obtaining apparatus according to the embodiment.

### DESCRIPTION OF THE EMBODIMENTS

According to the present invention, weighted optical characteristic information about the inside of an object is obtained to increase contrast in a photoacoustic image, the optical characteristic information being weighted on the basis of feature information about the object (hereinafter, also referred to as “object feature information”) obtained from an elastic wave signal acquired by transmission and reception of elastic waves. Herein, an elastic wave means an elastic wave (typically, an ultrasonic wave) transmitted from a probe. Furthermore, a photoacoustic wave means an elastic wave (typically, an ultrasonic wave) generated from a light absorber by irradiation of the light absorber with light. The feature information is information obtained by transmission and reception of elastic waves to and from the object and is an acoustic impedance, an amount of distortion (hereinafter, “distortion amount”), or an elastic modulus.

The above-described elastic wave signal is acquired using the straight-line propagation of an elastic wave inside the object. Specifically, a transmitted elastic wave is reflected in a local region inside the object, so that the elastic wave signal is acquired. Accordingly, information about the local region can be obtained. Object feature information obtained on the basis of the elastic wave signal acquired in the above-described manner can therefore be obtained as information about the local region. An image of the object feature information based on the elastic wave signal has a higher resolution than a photoacoustic image obtained by photoacoustic imaging in which incident light is diffused.

The object feature information represents a characteristic parameter (for example, a distortion amount) of an observation target (e.g., a tumor) which is hardly derived from optical characteristic information obtained by photoacoustic imaging.

Accordingly, optical characteristic information about the inside of an object is weighted on the basis of object feature information which offers high resolution as described above and represents a characteristic parameter of an observation target, thereby obtaining a photoacoustic image with high contrast between a region of interest and another region.

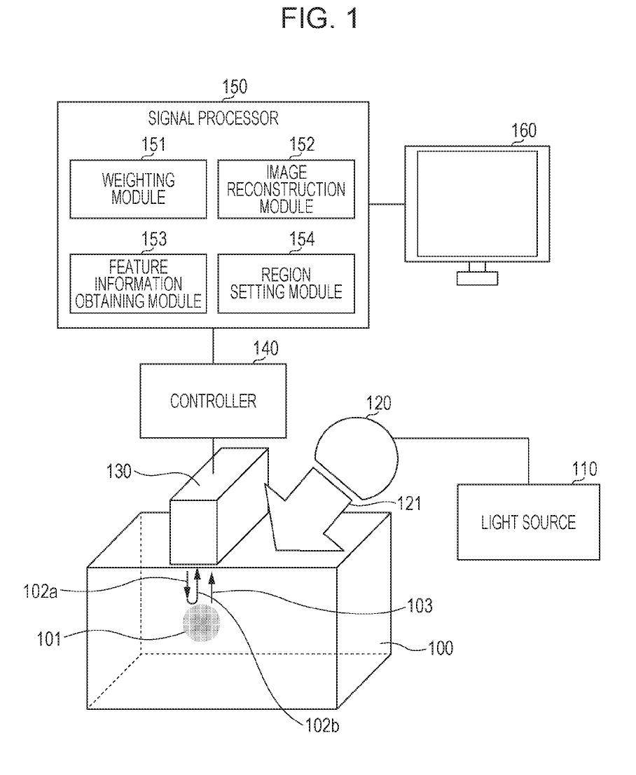

An object information obtaining apparatus according to an embodiment of the present invention will be described below with reference to FIG. 1. FIG. 1 schematically illustrates the object information obtaining apparatus according to the embodiment. As illustrated in FIG. 1, the object information obtaining apparatus includes a light source 110, an optical system 120, a probe 130, a controller 140, a signal processor 150 which serves as a signal processing unit, and a display 160 which serves as a display unit.

In this embodiment, the probe 130 has functions of an elastic wave transmitter that transmits an elastic wave to an object 100 and functions of an elastic wave receiver that receives an elastic wave propagated inside the object 100 and a photoacoustic wave.

The components will be described below.

### Object 100 and Light Absorber 101

The object 100 and a light absorber 101 will be described below, though they do not constitute the object information obtaining apparatus according to this embodiment. The object information obtaining apparatus according to this embodiment is mainly intended for diagnosis and chemical treatment follow-up of, for example, a malignant tumor or blood vessel disease in a human being or animal. A conceivable object is a living subject, specifically, a diagnosis target site, such as breast, neck, abdominal part, or rectum of a human or animal body.

A light absorber inside an object is an object having a relatively high absorption coefficient inside the object. For example, in the case where a human body is a target, examples of the light absorber include oxyhemoglobin, deoxyhemoglobin, a blood vessel in which much oxyhemoglobin or deoxyhemoglobin exists, and a malignant tumor including many new blood vessels. In addition, carotid wall plaque is included.

### Light Source 110

As regards the light source 110, a pulsed light source capable of generating pulsed light having a duration on the order of several nanoseconds to several microseconds may be used. Specifically, a pulse duration of approximately 10 nanoseconds is used to generate a photoacoustic wave with efficiency. A light emitting diode can be used instead of a laser light source. Any of various lasers, such as a solid laser, a gas laser, a dye laser, and a semiconductor laser, can be used. A wavelength at which light propagates into an object can be used. Specifically, a wavelength of 500 nm or more to 1200 nm or less can be used in the case where an object is a living subject.

### Optical System 120

Light emitted from the light source is typically guided through optical components, such as a lens and a mirror, to an object while being processed so as to have an intended light intensity distribution pattern through the components. An optical waveguide, such as an optical fiber, can be used to propagate light. The optical system includes a mirror that reflects light, a lens that converges or diverges light so as to change the pattern of light, and a diffuser that diffuses light. As regards the optical components, any optical component that allows an object to be irradiated with light, emitted from the light source, having an intended pattern may be used. Light diverged to some extent through the lens, rather than light converged therethrough, can be used from the viewpoints of assuring safety for a living subject and increasing a diagnosis region.

### Probe 130

The probe 130 is configured to detect an acoustic wave and convert the wave into an electrical signal which is an analog signal. Any detector capable of detecting an acoustic wave signal using, for example, piezoelectric phenomena, the resonance of light, or a change in capacitance may be used.

Furthermore, a probe which functions as an elastic wave transmitter and a probe which functions as an elastic wave receiver may be provided. Considering signal detection in the same region and space saving, the probe 130 may function as both the elastic wave transmitter and the elastic wave receiver.

The probe 130 may include a plurality of acoustic wave detecting elements arranged in an array.

### Controller 140

The object information obtaining apparatus according to this embodiment may include a controller that generates a transmission signal having a delay time and an amplitude appropriate for a position of interest or a direction of interest. The transmission signal is converted into an elastic wave by the probe 130 and the elastic wave is transmitted into an object.

The object information obtaining apparatus according to this embodiment may include the controller 140 that amplifies an electrical signal acquired through the probe 130 and converts the electrical signal, which is an analog signal, into a digital signal.

In the case where the probe 130 transmits and receives elastic waves through the acoustic wave detecting elements to acquire a plurality of electrical signals, the controller 140 can perform delay processing on the electrical signals in accordance with positions or directions in which the elastic waves are transmitted.

The controller 140 typically includes an amplifier, an A/D converter, and a field programmable gate array (FPGA) chip.

### Signal Processor 150

The signal processor 150 typically includes a work station in which signal processing, such as weighting or image reconstruction, is executed by pre-programmed software. For example, the software used in the work station includes a weighting module 151 that performs weighting which is characteristic signal processing of the present invention. The software further includes an image reconstruction module 152, a feature information obtaining module 153, and a region setting module 154 for setting a region of interest.

The modules may be arranged as individual hardware components. In this case, the modules can constitute the signal processor 150.

In photoacoustic imaging, an image based on a distribution of optical characteristics inside a living subject can be formed using a focused probe without image reconstruction. In such a case, it is unnecessary to perform signal processing using an image reconstruction algorithm.

In some cases, the controller 140 and the signal processor 150 may be combined. In such a case, object optical characteristic information about the object can be generated by hardware processing instead of by software processing performed in the work station.

### Display 160

The display 160 is a device to display optical characteristic information output from the signal processor 150. Typically, a liquid crystal display is used. The display 160 may be provided separately from the object information obtaining apparatus according to this embodiment.

A preferred embodiment of a method for obtaining object information using the object information obtaining apparatus illustrated in FIG. 1 will be described below.

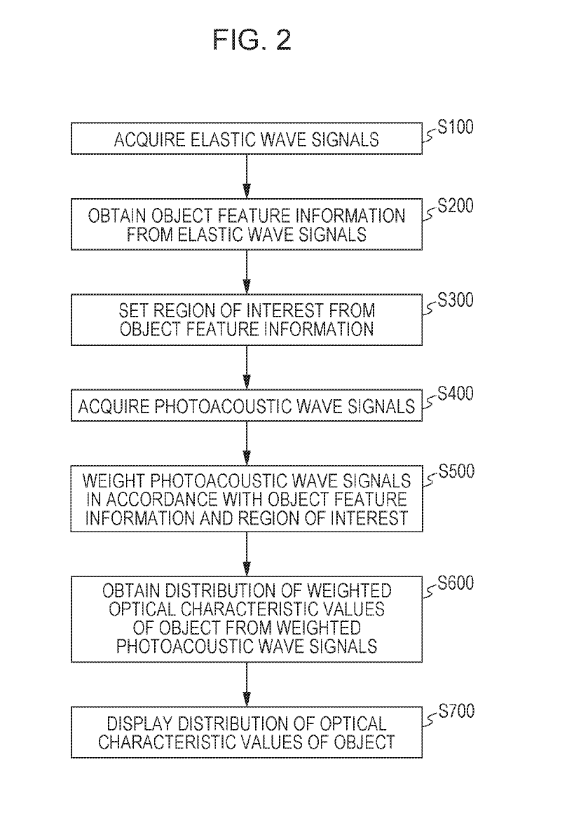

The method for obtaining object information according to this embodiment will be described with reference to FIG. 2.

### S100: Step of Acquiring Elastic Wave Signals

In this step, elastic waves are transmitted to and received from an object, thereby acquiring elastic wave signals.

The probe 130 transmits elastic waves 102a to the object 100 for flow velocity measurement, elastography measurement, or B-mode image measurement in order to obtain object feature information. In this case, the controller 140 transmits transmission signals having different delay times and different amplitudes for the acoustic wave detecting elements of the probe 130 depending on a position of a region of interest. The transmission signals are converted into the elastic waves 102a.

The transmitted elastic waves 102a are reflected inside the object, thereby generating echoes (elastic waves) 102b. The probe 130 receives the echoes 102b and outputs detection signals.

The controller 140 performs processing, such as amplification and A/D conversion, on the detection signals and stores the resultant signals as signal data in an internal memory of the controller 140. In this embodiment, it is a concept that elastic wave signals include the detection signals output from the probe 130 and the signals processed by the controller 140.

S200: Step of Obtaining Object Feature Information from Elastic Wave Signals

In this step, the feature information obtaining module 153 obtains object feature information from the elastic wave signals acquired in S100. A table of the obtained feature information is stored in an internal memory of the signal processor 150.

As regards feature information obtained from the elastic wave signals, any information from which an operator can determine the shape of an observation target by photoacoustic imaging may be used. Examples of the feature information include an acoustic impedance, a distortion amount, and an elastic modulus. Additionally, feature information may be appropriately selected depending on site or substance to be observed using photoacoustic wave signals.

For example, to distinguish a tumor region (new blood vessel region) from normal tissue, distortion amounts or elastic moduli may be obtained as feature information from the elastic wave signals acquired in S100. To obtain distortion amounts or elastic moduli, elastography measurement using elastic wave signals may be performed as disclosed in Journal of Medical Ultrasonic Volume 29, Number 3, 119-128, DOI: 10. 1007/BF02481234. Typically, a region (hard region) with a high elastic modulus is likely to be a malignant tumor and a region (soft region) with a low elastic modulus is unlikely to be a malignant tumor. Optical characteristic information calculated using photoacoustic waves substantially corresponds to a distribution of hemoglobin values and accordingly represents a blood vessel region and a distribution of tumor tissues where gathering of blood vessels is observed. The use of elastography measurement enhances the effectiveness of extracting the tumor region.

For example, to identify the boundary of biological tissue, acoustic characteristics, such as acoustic impedances, may be obtained as feature information from the elastic wave signals acquired in S100. In the case where the acoustic characteristics are obtained, B-mode image measurement using elastic wave signals may be performed. The inside of a cyst likely to be a tumor corresponds to an anechoic area of an image. Accordingly, regarding such an area as an observation target is effective in extracting a tumor.

S300: Step of Setting Region of Interest from Object Feature Information

In this step, a region setting unit sets a region of interest, serving as a region including a light absorber, from the object feature information obtained in S200. A table of the set region of interest is stored in the internal memory of the signal processor 150.

As regards a method of setting a region of interest, a method of setting a region of interest using a predetermined numerical range through the region setting module 154, serving as the region setting unit, included in the signal processor 150 or a method of setting a region of interest using a personal computer (PC) input device, serving as the region setting unit, through an operator may be used.

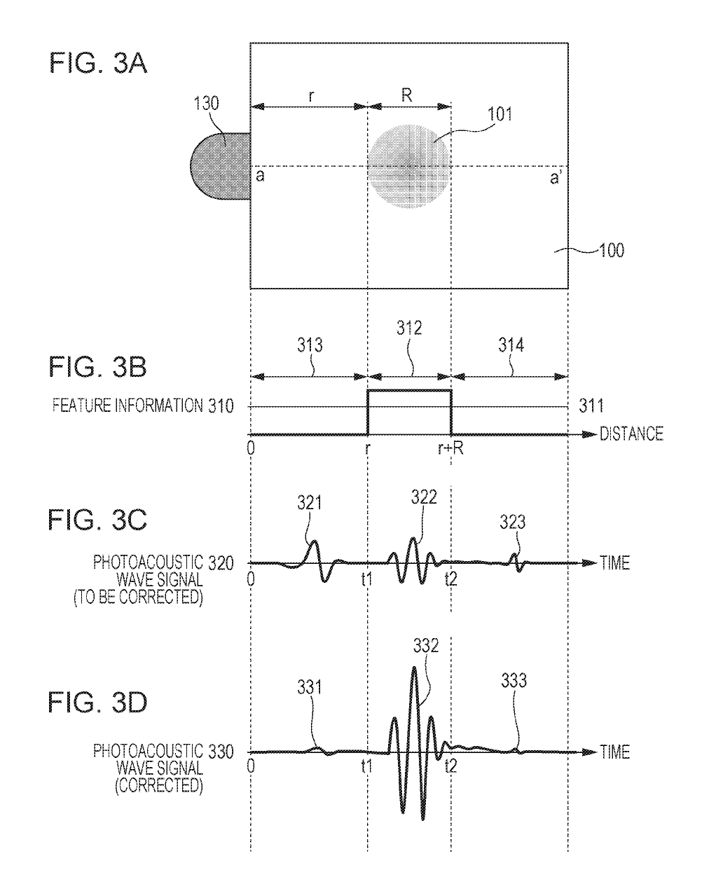

The method of setting a region within a predetermined numerical range as a region of interest through the region setting module 154 will now be described with reference to FIGS. 3A and 3B.

FIG. 3A is a diagram illustrating the object 100 and the probe 130 in FIG. 1 when viewed from the front. FIG. 3B illustrates feature information 310 in a position of a line a-a′ in FIG. 3A. FIG. 3B illustrates the value of feature information plotted against the distance from the probe 130. Referring to FIG. 3B, a region (between a distance r and a distance r+R from the probe 130) where the light absorber 101 exists has a higher feature information value than other regions.

In this step, for example, the region setting module 154 sets a threshold value 311 as illustrated in FIG. 3B. The region setting module 154 sets a region (region within the numerical range) in which the feature information 310 is greater than or equal to the threshold value 311 as a region 312 of interest. Whereas, the region setting module 154 sets regions (regions outside the numerical range) in which the feature information 310 is less than the threshold value 311 as other regions 313 and 314.

Specifically, the region setting module 154 sets a region where the feature information obtained in S200 is within the predetermined numerical range as a region of interest and sets a region where the feature information is outside the predetermined numerical range as another region.

As described above, in the case where feature information has a high value in a region where the light absorber 101 exists (for example, an observation target is a tumor having a high elastic modulus measured by elastography measurement), a region where feature information is greater than or equal to the threshold value 311 is set as a region of interest. Thus, a region including the light absorber 101 can be set as a region of interest.

In the case where feature information has a low value in the region where the light absorber 101 exists (for example, the observation target is a tumor having a small distortion amount measured by elastography measurement), values less than or equal to the threshold value may be within a numerical range.

As regards a method of setting a numerical range, the region setting module 154 can automatically set a numerical range using, for example, a technique of obtaining a threshold value for determination of a numerical range in which the degree of separation of measurement data by the discriminant analysis method is maximum. A threshold value for determination of the numerical range may be determined on the basis of the signal to system-noise ratio. Alternatively, the operator may specify any numerical range on the basis of the shape of a histogram of obtained feature information. The number of numerical ranges specified is not limited to one. A plurality of numerical ranges may be set.

The method of setting any region as a region of interest in an image of feature information with a PC input device, serving as the region setting unit, through the operator will now be described below.

First, an image of feature information is displayed on a monitor, serving as the display 160. Subsequently, the operator sets any region which is intended to be highlighted in an image of optical characteristic information as a region of interest in the displayed image of feature information. In this case, the operator may determine a start point and an end point using a mouse or sensors on a touch panel while viewing the displayed image of feature information and set a region between the start point and the end point as a region of interest.

The region setting unit may set a region within a predetermined numerical range as a region of interest and further set any portion of the set region as a region of interest.

### S400: Step of Acquiring Photoacoustic Wave Signals

In this step, photoacoustic waves generated by irradiation of the object with light are received, thereby acquiring photoacoustic wave signals.

Pulsed light 121 emitted from the light source 110 is applied to the object 100 through the optical system 120. The applied pulsed light 121 is absorbed by the light absorber 101, so that the light absorber 101 instantaneously expands, thereby generating photoacoustic waves 103. The probe 130 receives the photoacoustic waves 103 and outputs detection signals. The detection signals output from the probe 130 are subjected to processing, such as amplification and A/D conversion, by the controller 140 and the resultant signals are stored as detection signal data in the internal memory of the controller 140. In this embodiment, it is a concept that photoacoustic wave signals include the detection signals output from the probe 130 and the signals processed by the controller 140.

S500: Step of Weighting Photoacoustic Wave Signals in Accordance with Feature Information and Region of Interest

In this step, the weighting module 151 in the signal processor 150 weights the photoacoustic wave signals acquired in S400 on the basis of the feature information obtained in S200 and the region of interest set in S300. The weighted photoacoustic wave signals are stored in the internal memory of the signal processor 150.

Signal processing by the weighting module 151 will be described below with reference to FIGS. 3B to 3D.

FIG. 3C illustrates a photoacoustic wave signal 320 to be weighted by the weighting module 151. FIG. 3D illustrates a photoacoustic wave signal 330 weighted by the weighting module 151. FIGS. 3C and 3D illustrate the signal intensity of the photoacoustic wave signal plotted against detection time. The product of the speed of sound of a photoacoustic wave inside an object and detection time is a distance from the probe. Assuming that the sound speed of the photoacoustic wave inside the object is constant, a distance from the probe in FIG. 3B corresponds to detection time of the photoacoustic waves illustrated in FIGS. 3C and 3D. Specifically, the distance r in FIG. 3B corresponds to time t1 in FIGS. 3C and 3D and the distance r+R in FIG. 3B corresponds to time t2 in FIGS. 3C and 3D. The photoacoustic wave signal 320 of FIG. 3C includes signals 321 and 323 of the photoacoustic wave reflected multiple times on the surface of the probe 130. These signals 321 and 323 cause an artifact.

The weighting module 151 sets weighting factors for the photoacoustic wave signal 320 such that a weighting factor associated with the region 312 of interest is greater than those associated with the other regions 313 and 314, thus obtaining the weighted photoacoustic wave signal 330. In this case, as illustrated in FIG. 3D, the weighting factor associated with the region 312 of interest is greater than 1 and the weighting factors associated with the other regions 313 and 314 are less than 1.

The weighting module 151 may perform weighting such that a weighting factor associated with the region 312 of interest is less than 1 and weighting factors associated with the other regions 313 and 314 are greater than 1. Furthermore, the weighting module 151 may multiply the signal intensity of the photoacoustic wave signal associated with the other regions 313 and 314 by a weighting factor for reduction equal to a dynamic range.

In the case where feature information has a high value in the region where the light absorber 101 exists (for example, the observation target is a tumor having a high elastic modulus measured by elastography measurement), the weighting module 151 may use the value of the feature information as a weighting factor. In the case feature information has a low value in the region where the light absorber 101 exists (for example, the observation target is a tumor having a small distortion amount measured by elastography measurement), the weighting module 151 may use the inverse of the value of the feature information as a weighting factor.

Additionally, weighting may be performed using the ratio of a value of feature information to a certain value as a weighting factor. For example, the product of a value of feature information associated with the region of interest 312 and N and the product of a value of feature information associated with the other regions 313 and 314 and M can be used. The signal intensity of the photoacoustic wave signal associated with each region can be multiplied by the corresponding product.

Furthermore, the signal intensities of the photoacoustic wave signals associated with the entire region 312 of interest may be multiplied by the same weighting factor. The signal intensities of the photoacoustic wave signals associated with the entire other regions 313 and 314 may be multiplied by the same weighting factor. In this case, the photoacoustic wave signals associated with each region may be multiplied by a mean value of feature information associated with the region.

Additionally, a mean value of feature information associated with the region 312 of interest may be divided by a mean value of feature information associated with the other regions 313 and 314 and the signal intensity of the photoacoustic wave signal associated with the region 312 of interest may be multiplied by the quotient obtained in the above-described manner. Furthermore, the mean value of feature information associated with the other regions 313 and 314 can be divided by the mean value of feature information associated with the region 312 of interest and the signal intensity of the photoacoustic wave signal associated with the other regions 313 and 314 can be multiplied by the quotient obtained in the above-described manner. Such methods are particularly effective in measurement, such as elastography measurement, for identifying an observation target by measuring a relative difference in, for example, distortion amount or elastic modulus between a region of interest and another region.

As described above, in this step, weighting is performed, thus relatively reducing photoacoustic wave signals which are associated with the regions other than the region of interest and which cause an artifact or noise image.

S600: Step of Obtaining Weighted Optical Characteristic Information about Object from Weighted Photoacoustic Wave Signals

In this step, the image reconstruction module 152 in the signal processor 150 performs image reconstruction on the basis of the weighted photoacoustic wave signals acquired in S500, thus obtaining a weighted initial sound pressure distribution in the object (weighted optical characteristic information). The weighted initial sound pressure distribution is stored in the internal memory of the signal processor 150.

Since the image reconstruction module 152 performs image reconstruction using the weighted photoacoustic wave signals acquired in S500, the optical characteristic information obtained in this step is optical characteristic information weighted on the basis of the feature information. Specifically, since the image reconstruction is performed using the photoacoustic wave signals including relatively reduced photoacoustic wave signals which are associated with the regions other than the region of interest and which cause an artifact or noise image are relatively reduced, the optical characteristic information weighted such that the artifact or noise image is relatively reduced can be obtained.

The image reconstruction module 152 can use an image reconstruction algorithm, such as back projection in the time domain or Fourier domain which is typically used in tomography techniques. In the case where it may take much time for image reconstruction, an image reconstruction method, such as inverse problem analysis by repetitive processing, can also be used.

S700: Step of Displaying Optical Characteristic Information about Object

In this step, the weighted optical characteristic information obtained in S600 by the weighting module 151 is displayed as an image on the display 160. In this case, switching between a weighted image and an image to be weighted may be performed.

A program including the above-described steps may be executed by the signal processor 150 as a computer.

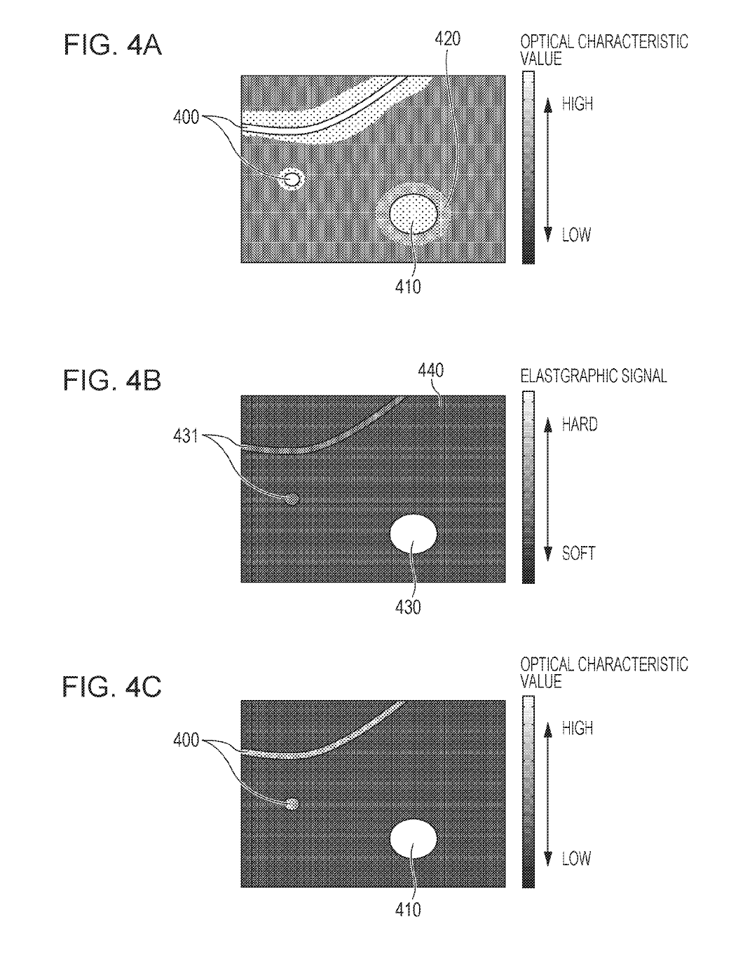

Examples of images obtained by the method of obtaining object information according to the present embodiment will be described with reference to FIGS. 4A to 4C.

FIG. 4A is a diagram illustrating optical characteristic information obtained by image reconstruction based on photoacoustic wave signals to be weighted, the signals being acquired from a living subject, serving as an observation target, including a tumor coated with new blood vessels. In FIG. 4A, the whiter a region, the higher its optical characteristic information value. In FIG. 4A, blood vessel images 400 and a tumor image 410 coated with new blood vessels are highlighted.

FIG. 4A illustrates an image obtained by image reconstruction based on the photoacoustic wave signals including photoacoustic wave signals associated with a region other than a region of interest. Accordingly, the optical characteristic information to be weighted illustrated in FIG. 4A includes an artifact 420 caused by false signals in addition to the blood vessel images 400 and the tumor image 410 coated with the new blood vessels.

FIG. 4B illustrates a distribution of distortions in the same observation target as that in FIG. 4A, the distribution being obtained by elastography measurement. Since tumor tissues are typically harder than other tissues as described above, characteristic signals can be obtained by elastography measurement. In addition, the tumor can be distinguished from new blood vessels by elastography measurement. In this case, regions 430 and 431 of interest for elastography and a region 440 other than the regions of interest for elastography are set using the method described in S300.

The photoacoustic wave signals, which are to be weighted, associated with the regions 430 and 431 of interest for elastography and the other region 440 are weighted using the method described in S500. The weighted photoacoustic wave signals acquired in this manner are used for image reconstruction, thus obtaining weighted optical characteristic information illustrated in FIG. 4C.

The comparison between the images of FIGS. 4A and 4C demonstrates that the artifact 420 caused by the false signals exists in FIG. 4A and that the artifact is reduced and the blood vessel images 400 and the tumor image 410 can be easily identified in FIG. 4C. In addition, the blood vessel images 400 can be easily distinguished from the tumor image 410 in FIG. 4C.

According to the method of obtaining object information as described in this embodiment, photoacoustic wave signals are weighted and the weighted photoacoustic wave signals are used for image reconstruction, thus obtaining weighted optical characteristic information. In the weighted optical characteristic information obtained in this manner, an artifact or noise image in a region other than a region of interest is relatively reduced. Consequently, a photoacoustic image with high contrast between the region of interest and the other region can be obtained.

Furthermore, according to an object information obtaining method of a modification of the embodiment, photoacoustic wave signals to be weighted can be used for image reconstruction, thus obtaining optical characteristic information as illustrated in FIG. 4A. The obtained optical characteristic information can be weighted in the same way as in S500, thus obtaining weighted optical characteristic information. Specifically, although the photoacoustic wave signals are weighted in the foregoing embodiment, optical characteristic information can be similarly weighted according to the modification. Accordingly, an artifact or noise image in a region other than a region of interest can be relatively reduced. Thus, a photoacoustic image with high contrast between the region of interest and the other region can be obtained.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An apparatus for obtaining object information, comprising:
a signal processing unit configured to obtain optical characteristic information about an object on the basis of a photoacoustic wave signal acquired by reception of a photoacoustic wave generated by irradiation of the object with light and obtain weighted optical characteristic information about the object on the basis of feature information about the object obtained by elastography measurement or B-mode image measurement using an elastic wave signal acquired by transmission and reception of elastic waves to and from the object.
